(19) United States
(12) Patent Application Publication
(10) Pub. No.: US 2026/0338399 A1
(43) Pub. Date: Oct. 29, 2026

(54) AIR PURIFICATION SYSTEM

(71) Applicant: Dyson Technology Limited, Malmesbury, Wiltshire (GB)

(72) Inventors: Niloufar ZARINABAD NOORALIPOUR, Birmingham (GB); Benjamin James HOVELL, Tetbury (GB)

(21) Appl. No.: 19/106,279

(22) PCT Filed: Aug. 8, 2023
(86) PCT No.: PCT/IB2023/057995

§ 371 Date: Feb. 25, 2025

(30) Foreign Application Priority Data
Aug. 26, 2022 (GB) 2212429.1

(51) Int. Cl. F24F 11/30; F24F 8/108; F24F 8/80; F24F 11/63; F24F 110/50; F24F 120/20

(52) U.S. Cl. CPC F24F 11/30; F24F 8/108; F24F 8/80; F24F 11/63; F24F 2110/50; F24F 2120/20

(57) ABSTRACT

An air purification system including: an air purification device; and a network server. The air purification device includes: an air purification unit configured to be operated to purify air in the vicinity of the air purification device; and a communication interface, wherein the air purification device is configured to communicate with the network server via the communication interface. The air purification device is configured to operate the air purification unit according to a control algorithm that uses sensor data representative of the content of air in the vicinity of the air purification device as an input. The network server is configured to update the control algorithm to change how the air purification device operates the air purification unit according to sensor data representative of the content of air in the vicinity of the air purification device.

### FIELD OF THE INVENTION

The present invention relates to an air purification system.

### BACKGROUND

In order to maintain or improve air quality in an indoor environment, it is known to use an air purification device. An air purification device typically includes an air purification unit configured to be operated to purify air in the vicinity of the air purification device.

The air purification unit may, for example, include a fan assembly to draw air into the air purification device, and a purification assembly configured to purify air drawn into the air purification device by the fan.

The purification assembly may be configured to purify air (drawn into the air purification device by the fan) by, for example, reducing the concentration of one or more chemical elements, chemical compounds, classes of chemical compounds and/or other materials (e.g. particulate matter) present in the air passing through the device. For example, the chemical compounds may include volatile organic compounds (VOCs), nitrous oxides, formaldehyde, carbon dioxide, carbon monoxide. The one or more other materials may include smoke and/or particulate matter. The one or more chemical elements may include lead particles. The purification assembly may include one or more filtration elements.

It is known for an air purification device to include sensor unit configured to provide the sensor data representative of the content of air in the vicinity of the air purification device, and for the air purification system of the air purification device to be controlled according to the sensor data provided by the sensor unit.

The present inventors have observed that a problem with known air purification devices is that the control algorithms used to control air purification devices according to sensor data are quite simplistic, and tend to involve the device switching on when measured values exceed a threshold. Moreover, these algorithms tend to be fixed, and do not change in accordance with user preference or newly identified harmful compounds. The present inventors have observed that this results in users having little control over what odours are removed by their air purification devices.

The present invention has been devised in light of the above considerations.

WO2019/074256 discloses an odour mitigation system for a vehicle that can detect smells or odours in a vehicle using a sensor such as an e-nose. Users of the vehicle can indicate whether or not a detected odour is agreeable or disagreeable, enabling a mitigation approach for disagreeable or undesirable odours to be recommended.

The present invention has been devised in light of the above considerations.

### SUMMARY OF THE INVENTION

In a first aspect, the present invention provides: An air purification system including:
an air purification device; and
a network server;
wherein the air purification device includes:
an air purification unit configured to be operated to purify air in the vicinity of the air purification device; and
a communication interface, wherein the air purification device is configured to communicate with the network server via the communication interface;
wherein the air purification device is configured to operate the air purification unit according to a control algorithm that uses sensor data representative of the content of air in the vicinity of the air purification device as an input;
wherein the network server is configured to update the control algorithm to change how the air purification device operates the air purification unit according to sensor data representative of the content of air in the vicinity of the air purification device.

In this way, the network server can change how the air purification device responds to sensor data, e.g. so as to improve the performance of the air purification device, e.g. by changing to target newly identified harmful compounds.

In some examples, the network server may be configured to update the control algorithm based on sensor data associated with a chemical element, chemical compound, class of chemical compound and/or other material that has been identified as harmful to users. For example, an entity in control of the network server (e.g. a supplier of the air purification device) may have identified a new compound or class of compounds identified as harmful, and may update the control algorithm such that the air purification device operates so as to reduces the amount of that compound or class of compounds present in the vicinity of the air purification device.

In some examples, the network server may be configured to update the control algorithm based on sensor data associated with user input data indicative of user preference regarding the content of air in the vicinity of the air purification device.

In this way, the control algorithm can be updated in a manner compliant with user preferences.

In some examples, the air purification device may include a user interface, and the user input data may be derived from user input provided via the user interface.

In some examples, the air purification device may include the user interface. In other examples, the user interface may be included in a device (e.g. a mobile device such as a smartphone) connected to the air purification device (e.g. via the communication interface of the air purification device).

The user interface may be configured to permit a user to provide a “positive opinion” input indicating that the user has a positive opinion regarding an odour of air in the vicinity of the device at a time at which the “positive opinion” input is provided. The user input data may include data which associates the positive opinion of the user with sensor data representative of the content of air in the vicinity of the air purification device at the time the “positive opinion” input was provided.

The user interface may be configured to permit a user to provide a “negative opinion” input indicating that the user has a negative opinion regarding an odour of air in the vicinity of the device at a time at which the “negative opinion” input is provided. The user input data may include data which associates the negative opinion of the user with sensor data representative of the content of air in the vicinity of the air purification device at the time the “negative opinion” input was provided.

The user interface may be configured to permit a user to provide a “neutral opinion” input indicating that the user has a neutral opinion (i.e. neither a positive opinion nor a negative opinion) regarding an odour of air in the vicinity of the device at a time at which the “neutral opinion” input is provided. The user input data may include data which associates the neutral opinion of the user with sensor data representative of the content of air in the vicinity of the air purification device at the time the “neutral opinion” input was provided.

In some examples, there may be only one type of “positive opinion” input (e.g. corresponding to a thumbs up button) and only one type of “negative opinion” input (e.g. corresponding to a thumbs down button). However, in other examples, the user interface may be configured to permit a user to provide further “opinion” inputs. For example, a five-point scale of “strongly negative”, “negative”, “neutral”, “positive”, “strongly positive” may be provided. Alternative gradations of “positive opinion” and/or “negative opinion” may also be used.

The association of an input (indicating that the user has a given opinion regarding an odour of air in the vicinity of the device at a time at which the input is provided) with sensor data may be achieved by any appropriate means known to the skilled person.

In some examples, wherein the sensor data used to update the control algorithm includes pooled sensor data associated with multiple users, wherein the multiple users include at least one user of an air purification device other than the air purification device.

In this way, sensor data from multiple users of multiple air purification devices can be leveraged to improve the performance of the air purification device, without necessarily relying on input provided by a user of the air purification device.

In some examples, the multiple users may belong to a limited population group who share at least one characteristic with a user of the air purification device.

In this way, sensor data from multiple users can be leveraged to improve the performance of the air purification device in a manner that may reflect the user's preferences (by virtue of sharing the at least one characteristic), without necessarily relying on that user to provide input regarding their preferences.

By way of example, the members of the population group may share a geographical location characteristic, e.g. the members may belong to a same geographical region (e.g. country, group of countries, continent). This may be useful if people within that geographical region share preferences regarding desirable/undesirable smells.

By way of example, the members of the population group may share an age characteristic, e.g. the members may belong to a same age bracket. This may be useful if people within that age bracket share preferences regarding desirable/undesirable smells.

By way of example, the members of the population group may share a gender characteristic, e.g. the members may belong to a same gender category (e.g. male, female, gender neutral, cisgender, transgender). This may be useful if people within that gender category share preferences regarding desirable/undesirable smells.

By way of example, the members of the population group may share a user preference characteristic regarding the content of air in the vicinity of the air purification device. For example, the members of the population group may all have similar preferences regarding smells they like and/or dislike (without necessarily belonging to the same geographical region, age bracket or gender).

The air purification system may be configured to categorise a user of the air purification device into the population group (who share a user preference characteristic regarding the content of air in the vicinity of the air purification device) based on user input in response to the content of air in the vicinity of the air purification device.

The air purification system may be configured to a use a machine learning model to form the population group and to categorise the user into the population group. The machine learning model used to form the population group based on shared user preference characteristics may be a clustering algorithm.

In some examples:
the control algorithm may include a machine learning model; and
the network server may be configured to update the control algorithm by updating the machine learning model using training data.

The training data may include sensor data associated with user input data indicative of user preference regarding the content of air in the vicinity of the air purification device.

The training data may include pooled sensor data associated with multiple users, wherein the multiple users include at least one user of an air purification device other than the air purification device.

The training data may comprise sensor data associated with a chemical elements, a chemical compound or a class of chemical compounds that has been identified as harmful to users (e.g. by an original equipment manufacturer—OEM).

In some examples, the machine learning model is located at the network server, and the network server is configured to instruct the air purification unit to operate according to an output of the machine learning model.

In some examples, the machine learning model is located at the air purification device, and the network server is configured to update the control algorithm to change how the air purification device operates the air purification unit by updating the machine learning model located at the air purification device.

In some examples where the machine learning model is located at the air purification device, the network server may be configured to update the control algorithm to change how the air purification device operates the air purification unit by sending an updated machine learning model for use by the air purification device, to the air purification device.

In some examples where the machine learning model is located at the air purification device, the network server may be configured to update the control algorithm to change how the air purification device operates the air purification unit by sending training data to the air purification device so that the air purification device updates the machine learning model using the training data.

In some examples, operating the air purification unit according to the control algorithm comprises the air purification system:
determining, from the sensor data, whether the air in the vicinity of the air purifying device contains a chemical element, a chemical compound or a class of chemical compounds that is undesirable; and
operating the air purification unit to purify air in the vicinity of the air purification device if it is determined that the air in the vicinity of the air purifying device contains a chemical element, a chemical compound or a class of chemical compounds that is undesirable.

The chemical element, the chemical compound or the class of chemical compounds may be determined to be undesirable based, at least in part, on user inputs indicative of user preferences regarding the content of air in the vicinity of the air purification device.

The chemical element, the chemical compound or the class of chemical compounds may be determined to be undesirable based, at least in part, on the chemical element, the chemical compound or a class of chemical compounds being identified (e.g. by a manufacturer of the device) as harmful to users.

In some examples, operating the air purification unit according to the control algorithm comprises the air purification system:
determining, from the sensor data produced, whether the air in the vicinity of the air purifying device contains a chemical element, a chemical compound or a class of chemical compounds that is desirable;
terminating operation of the air purification unit if it is determined that the air in the vicinity of the air purifying device contains a chemical element, a chemical compound or a class of chemical compounds that is desirable.

The chemical element, the chemical compound or the class of chemical compounds may be determined to be desirable based, at least in part, on user inputs indicative of user preferences regarding the content of air in the vicinity of the air purification device.

In some examples, the air purification unit may include a fan assembly to draw air into the air purification device, and a purification assembly configured to purify air drawn into the air purification unit by the fan assembly.

In some examples, the air purification device may be for use in a building.

In some examples, the air purification device may be wearable. The air purification device may be a wearable mask. If the air purification device is a wearable mask, the sensor data representative of the content of air in the vicinity of the air purification device may be obtained by a suitably located sensor on the mask.

In some examples, the air purification system may include a sensor unit configured to provide the sensor data representative of the content of air in the vicinity of the air purification device.

In some examples, the air purification device may comprise the sensor unit.

In some examples, the air purification system may comprise a sensor unit that is separate to the air purification device. The separate sensor unit may be configured to communicate sensor data to the air purification device via the communication interface. The separate sensor unit could, for example, be a standalone sensor unit, or could be incorporated into another device, such as a wall clock or smartwatch.

In some examples, the sensor unit may include an e-nose sensor.

In some examples, the sensor unit may include includes a VOC (Volatile organic compound) sensor or a VIC (Volatile inorganic compound) sensor.

In some examples, the sensor unit may include a particle counter.

In some examples, the sensor unit may include a temperature sensor.

In some examples, the sensor unit may include a humidity sensor.

In some examples, the sensor unit may include a pressure sensor.

In a second aspect of the invention, there may be provided an air purification device suitable for use (e.g. configured to be used) in an air purification system according to the first aspect of the invention.

The invention includes the combination of the aspects and preferred features described except where such a combination is clearly impermissible or expressly avoided.

### SUMMARY OF THE FIGURES

Embodiments and experiments illustrating the principles of the invention will now be discussed with reference to the accompanying figures in which:

FIG. 1 shows an example air purification system.

FIGS. 2A-C shows an example air purification device for use in the air purification system of FIG. 2

FIGS. 3-5 show different examples of how the air purification device 110 of the air purification system 100 of FIG. 1 may use a control algorithm that includes a machine learning model.

### DETAILED DESCRIPTION OF THE INVENTION

Aspects and embodiments of the present invention will now be discussed with reference to the accompanying figures. Further aspects and embodiments will be apparent to those skilled in the art. All documents mentioned in this text are incorporated herein by reference.

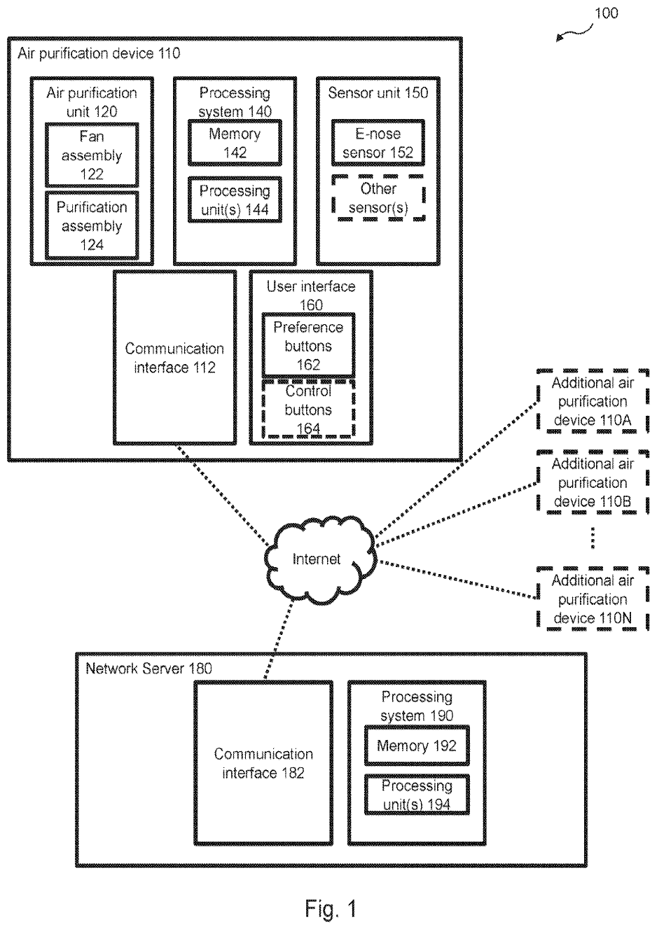

FIG. 1 shows an example air purification system 100.

The air purification system 100 includes an air purification device 110 and a network server 180.

The air purification device 110 includes an air purification unit 120 configured to be operated to purify air in the vicinity of the air purification device 110, and a communication interface 112, wherein the air purification device 110 is configured to communicate with the network server 180 via the communication interface 112.

The communication interface 112 may, for example, be a wireless network interface, e.g. configured to communicate with the internet via one or more known wireless networking protocols.

The air purification unit 120 includes a fan assembly 122 to draw air into the air purification device 120, and a purification assembly 124 configured to purify air drawn into the air purification unit 120 by the fan assembly 122.

The air purification device 110 also includes a processing system 140, which may include, for example, a memory 142 and one or more processing units 144.

In this example, the air purification device 110 includes a sensor unit 150 configured to provide sensor data representative of the content of air in the vicinity of the air purification device 110.

In other examples, the sensor unit 150 may be separate to the air purification device 110. The separate sensor unit may be configured to communicate sensor data to the air purification device 110 via the communication interface 112. The separate sensor unit could, for example, be a standalone sensor unit, or could be incorporated into another device, such as a wall clock, mobile device or smartwatch.

In this example, the sensor unit 150 includes an electronic nose (“e-nose”) sensor 152 for producing sensor data representative of the chemical content of air in the vicinity of the air purification device. An e-nose may be used to detect chemical compounds of interest (including, but not necessarily limited to, chemical compounds which can be smelled by a human nose), and may be used as an electronic substitute for a human nose.

An e-nose sensor typically comprises a plurality (e.g. 5-64) of individual sensors (pixels) which are used to produce sensor data representative of the chemical content of air in the vicinity of the e-nose (and hence in the vicinity of the air purification device, if the e-nose is in the vicinity of the air purification device).

The e-nose sensor may produce sensor data in the form of raw signal data taken from the individual sensors in the e-nose, or in the form of data which has been derived from the raw signal data. If a particular chemical compound is present in air in the vicinity of the e-nose sensor (and hence present in the vicinity of the air purifying device), then sensor data produced by the e-nose at that time may include a signature indicative of that chemical compound.

Typical sensor types for an e-nose may include electrical sensors (e.g. chemiresistive sensors which measure a change in resistance or impedance), mechanical sensors (e.g. quartz crystal microbalance which measure a change in resonant frequency), or optical sensors (which measure change in fluorescence, light conductivity, or colour for a chemically responsive component).

The individual sensors within the e-nose sensor 152 may be selective, such that the array of sensors responds to different chemicals or groups of chemicals. Each individual sensor may be configured to be sensitive to a single compound (e.g. formaldehyde) or a wide group, e.g. volatile organic compounds (VOCs). In particular, the e-nose sensor 152 may include a selective membrane (e.g. molecular size/shape selective membrane) covering one or more of the sensors to limit which molecules can impinge on each sensor. Additionally or alternatively, the sensors themselves may be respectively tuned such that each sensor only responds to certain molecules, chemicals, or groups thereof.

In some examples, the e-nose sensor 152 may be a silicon photonics based e-nose sensor with an array of Mach Zehnder Interferometers. An example of such an e-nose sensor is the “NeOse advance” available from Aryballe (https://aryballe.com/our-products/device-solutions/).

In other examples, the e-nose sensor 152 may be a chemiresistive array. An example of such an e-nose sensor is SafeScent from Stratuscent (https://www.stratuscent.com/safescent).

Of course, other types of e-nose sensors are available, as discussed above.

The sensor unit 150 may include one or more other sensors such as a particle counter, a temperature sensor, a humidity sensor, a pressure sensor.

In this example, the air purification device 110 includes a user interface 160 configured to allow a user to provide user input data indicative of user preference regarding the content of air in the vicinity of the air purification device.

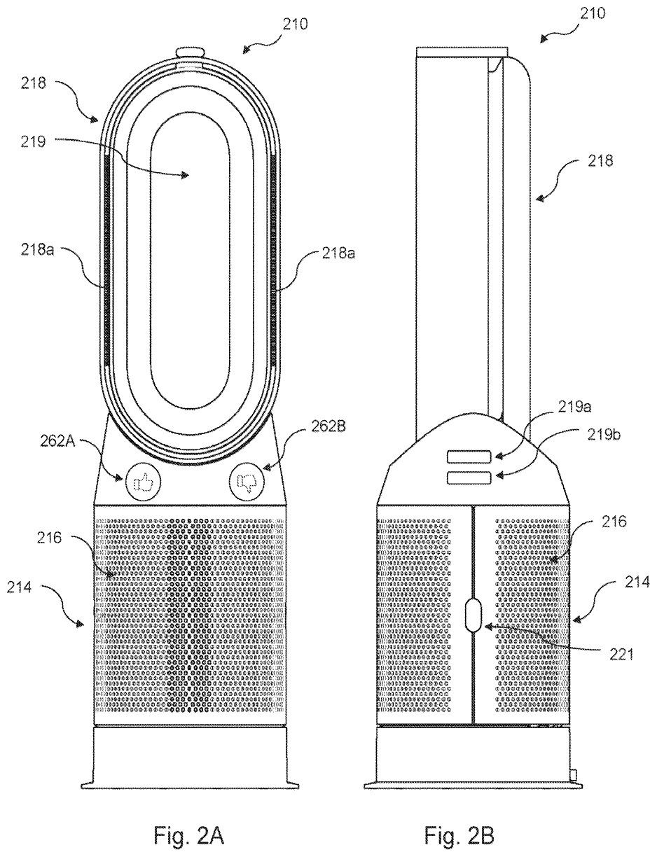

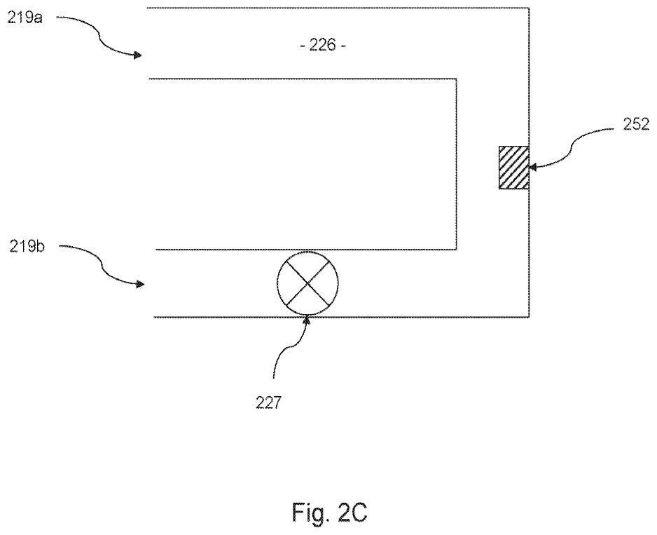

In this example, the user interface 160 includes preference buttons 162, which include a “thumbs up” button (see element 262A in FIG. 2A) and a “thumbs down” button (see element 262B in FIG. 2A).

The “thumbs up” button is configured to permit a user to provide a “positive opinion” input by pushing the “thumbs up” button. This “positive opinion” input indicates that the user has a positive opinion regarding an odour of air in the vicinity of the device at a time at which the “thumbs up” button is pushed. The user input data may include data which associates the positive opinion of the user with sensor data representative of the content of air in the vicinity of the air purification device at the time the “positive opinion” input was provided.

The “thumbs down” button is configured to permit a user to provide a “negative opinion” input by pushing the “thumbs down” button. This “negative opinion” input indicates that the user has a negative opinion regarding an odour of air in the vicinity of the device at a time at which the “thumbs down” button is pushed. The user input data may include data which associates the negative opinion of the user with sensor data representative of the content of air in the vicinity of the air purification device at the time the “positive opinion” input was provided.

The user interface 160 may include additional control buttons 164 for initiating other operations to be performed by the air purification device (e.g. turning power on/off or turning wireless connectivity on/off).

In other examples (not shown), the user interface may be provided by a separate device (e.g. a mobile device such as a mobile phone) connected to the air purification device 110 via the communication interface 112 of the air purification device 110.

The network server 180 includes a communication interface 182 and a processing system 190.

The communication interface 182 may, for example, be a wired network interface, e.g. configured to communicate with the internet via one or more known wired networking protocols.

The processing system 190 of the network server 180 may include, for example, a memory 192 and one or more processing units 194.

Although FIG. 1 shows the air purification device 110 and the network server 180 communicating via the internet, it would also be possible for the air purification device 110 and the network server 180 to communicate directly, i.e. without involvement of the internet.

The air purification system 100 may include one or more additional air purification devices 110A, 110B . . . 110N, which may each include components corresponding to the air purification device 110.

FIGS. 2A-B shows an example air purification device 210 for use (e.g. as the air purification device 110) in the air purification system 100 of FIG. 1.

The air purification device 210 comprises a body 214 comprising an air inlet 216 through which a primary airflow enters the body 214, at least one removable purification assembly (not shown) mounted in the body, and a nozzle 218 mounted on an air vent/opening through which the primary airflow exits the body 214. A primary fan assembly (not shown) is provided within the body 214 to draw air from the air inlet 216 to the nozzle 218 through the purification assembly. The nozzle 218 comprises air outlets 218a, 218b for emitting the primary airflow from the air purification device 210. The nozzle 218 also defines a central/inner opening/bore 219 through which air from outside the air purification device 210 is drawn by the primary airflow emitted from the outlets 218a, 218b. The nozzle 218 therefore forms a loop that extends around and surrounds the bore 219.

The purification assembly includes one or more filtration elements (e.g. filters) which function to reduce the concentration of one or more chemical compounds and/or other materials (e.g. particulate matter), when the primary fan assembly is operated. In particular the filtration elements contain one or more filter media to effect the filtration upon air drawn into the air purification device 210 by the primary fan assembly.

The primary fan assembly and purification assembly together form an air purification system configured to be operated to purify air in the vicinity of the air purification device. In this example, the air purification system is operated by operating the primary fan assembly to draw air from the air inlet 216 to the nozzle 218 through the purification assembly.

The features of the air purification device 210 relating to the fan and the purification of air is described in greater detail in GB2568979A, and therefore need not be repeated here.

FIG. 2C shows a sensor airflow channel 226 within the air purification device 210 of FIGS. 2A-B.

The sensor airflow channel 226 extends from a sensor airflow inlet 219a to a sensor airflow outlet 219b

The sensor airflow channel 226 includes a secondary fan assembly 227 which is configured to be operated to cause a secondary airflow to flow through the sensor airflow channel from the sensor airflow inlet 219a to a sensor airflow outlet 219b. The secondary fan assembly 227 is configured to be operated independently of the primary airflow discussed above, which allows the secondary airflow to flow even when the primary fan assembly is not operated.

In this example, the air purification device 210 of FIGS. 2A-B includes an e-nose sensor 252, which is configured to provide sensor data representative of the content of air in the vicinity of the air purification device.

The e-nose sensor 252 may be as discussed above in relation to FIG. 1.

The airflow in the sensor airflow channel 226 is preferably ambient air, which has not been filtered/heated/cooled by the air purification device. To achieve this, the sensor airflow inlet 219a and the sensor airflow outlet 219b may be appropriately located on an external surface of the body of the air purification device 210, such that purified/heated/cooled air from the primary airflow does not pass over the sensor airflow inlet 219a and the sensor airflow outlet 219b. In the example of FIG. 2B, the sensor airflow inlet 219a and the sensor airflow outlet 219b are located on a side of a body portion of the air purification device 210, in order to achieve this effect.

In some examples, the e-nose sensor 252 may be the only sensor in the air purification device 210, in which case the e-nose sensor 252 may (alone) form the sensor unit of the air purification device 210. In other examples, one or more other sensors may be incorporated in the air purification device 210, with the e-nose sensor and the other sensor(s) together form the sensor unit of the air purification device 210. The one or more other sensors may be included in the sensor airflow channel 226 as shown in FIG. 2C. or in additional sensor airflow channels (not shown in FIGS. 2A-C).

Unlike the air purification device described in GB2568979A, the air purification device 210 shown in FIGS. 2A-B includes preference buttons in the form of a thumbs up button 262A and a thumbs down button 262B. As discussed in more detail above, these preference buttons allow a user to provide user input data indicative of user preference regarding the content of air in the vicinity of the air purification device.

The air purification device 210 also includes a communication interface (not shown in FIGS. 2A-C) as discussed above, and an on/off power button.

Referring back now to FIG. 1, the control algorithm according to which the air purification device 110 operates may include a machine learning model. The network server 180 may be configured to update the control algorithm by updating the machine learning model using training data.

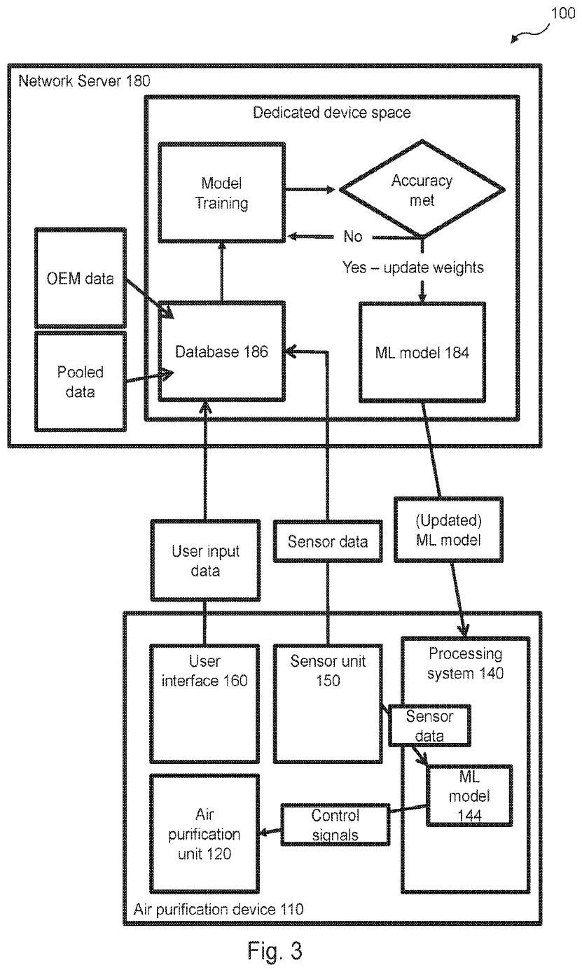

FIGS. 3-5 show different examples of how the air purification device 110 of the air purification system 100 of FIG. 1 may use a control algorithm that includes a machine learning model.

In the example shown in FIG. 3, a machine learning model 144 used as part of the control algorithm is located at the air purification device 110, and the network server 180 is configured to update the control algorithm to change how the air purification device 110 operates the air purification unit 120 by updating the machine learning model 144 located at the air purification device.

The machine learning model 144 used here is a model which is configured to make a determination of whether or not to operate the air purification unit 120 of the air purification device 110, based on sensor data representative of the content of air in the vicinity of the air purification device. The model is located at the processing system 140 of the air purification device 110, where it is stored in the memory 142.

In this example, the processing system 140 of the air purification device is configured to send control signals to instruct the air purification unit 120 to operate (or not) according to an output of the machine learning model 144.

The network server 180 is configured to update the control algorithm by updating the machine learning model 144 using training data.

In this example, the network server 180 is configured to update the machine learning model 144 located at the air purification device 110 by generating an updated version of the machine learning model 184 at the server 180, and then communicating this updated version of the machine learning model 184 to the processing system of the air purification device, where it is stored in the memory 142 as a new version of the machine learning model 144, to replace the previous version of the machine learning model 144.

In some examples, a new version of the machine learning model 144 may be added to the air purification device through a separate communication channel to that used to transmit sensor data to the network server. For example, a new version of the machine learning model may be made available for download to a removable storage device that can then be used to transfer the new version of the machine learning model to the air purification device. This may help improve the resilience of the air purification device operation against network connection unreliability.

The network server 180 is configured to generate the updated machine learning model 184 using training data.

In detail, a training database 186 at the network server 180 stores training data which is used to train the machine learning model 184 In this example, the training data includes:
(i) User input data and sensor data associated with the user input data communicated from the air purification device 110
(ii) Pooled sensor data associated with the user input data of multiple users. Thus the pooled sensor data may include “positive opinion” or “negative opinion” inputs provided by those multiple users at other air purification devices, such as devices 110A-N shown in FIG. 1. In some examples, the pooled sensor data may include only one type of “positive opinion” input (corresponding to the thumbs up button 262A shown in FIG. 2A) and only one type of “negative opinion” input (corresponding to the thumbs down button 262B shown in FIG. 2B). However, in other examples, more than one type of “positive opinion” input or “negative opinion” input (e.g. different gradations of positive and/or negative opinions) may be used. For example, a five-point scale of “strongly negative”, “negative”, “neutral”, “positive”, “strongly positive” may be provided.
(iii) Original equipment manufacturer (“OEM”) data from a manufacturer of the air purification devices 110, 110A-N. The OEM data may include sensor data associated with a chemical element, chemical compound, or a class of chemical compounds that has been identified as harmful to users.

This training data is used to train or re-train the machine learning model 184 at the “Model Training” functional block, such that:
(A) the machine learning model 184 is trained to determine that the air purification unit should be turned on when sensor data associated with a “negative opinion” from the user of the air purification device 110 or one of the multiple users associated with the pooled data is provided as an input to the machine learning model; and/or
(B) the machine learning model 184 is trained to determine that the air purification unit should be turned on when sensor data associated with a chemical element, a chemical compound or a class of chemical compounds that has been identified as harmful to users in the OEM data, is provided as an input to the machine learning model; and/or
(C) the machine learning model is trained to determine that the air purification unit should be turned off when sensor data associated with a “positive opinion” from the user of the air purification device 110 or one of the multiple users associated with the pooled data is provided as an input to the machine learning model

Training a machine learning model in this way would be well within the capability of a skilled person based on the disclosure herein, e.g. using known machine learning training techniques and libraries. For example, the machine learning model 144 (and thus model 184) may appropriately be chosen as a decision tree algorithm, which can be used with individual static values of each sensor element within the e-nose at different instances as the data points. The above training can be posed as a non-linear step-based problem where decision can be made through series of conditions and these conditions are learned using ground truth data. A decision tree is the simplest algorithm that can perform condition-based learning and it may require less computational effort compared to other non-linear methods. The machine learning model may be constructed using standard machine learning libraries, such as the scikit learn python library, which may be licensed under permissive simplified BSD license which allows it to use in commercial applications.

After (re-)training, the (re-)trained model is then tested to see if it has a required level of accuracy, see functional block 188. If the (re-)trained model has a required level of accuracy, then the weights of the machine learning model 184 are updated accordingly, so as to generate an updated machine learning model 184. This updated version of the machine learning model 184 is then communicated to the air purification device as described above.

If the (re-)trained model lacks a required level of accuracy, then the model requires further training. This re-training may optionally involve obtaining more data at the database, which can be used as part of the retraining.

Note here that the machine learning model doesn't need to identify chemicals or smells based on the sensor data. Rather, the machine learning model has been trained to operate the air purification unit (or not) based on sensor data and on how it has been trained using the training data. In other words, the machine learning model can determine that a chemical or smell is desirable undesirable without requiring direct and/or specific identification of that chemical or odour.

In some examples, the training database may include metadata regarding the air purification device 110, such as device model, software version, IP address location. This metadata may, for example, influence how sensor data from the device is interpreted. IP address location may indicate the location of the air purification device, which can be used to pool data from geographically similar users as discussed further below.

Preferably, the multiple users (with whom the pooled sensor data is associated) belong to a limited population group who share at least one characteristic with a user of the air purification device.

By way of example, the members of the population group may share a geographical location characteristic, age characteristic, gender characteristic and/or a user preference characteristic.

The geographical location characteristic (e.g. country) may be provided by a user during a process of registering their air purification device, or via an IP address of the air purification device (since a geographical location can be derived from an IP address, albeit this geographical location may not always be accurate, e.g. since the user may use a VPN).

In this way, sensor data from multiple users can be leveraged to improve the performance of the air purification device in a manner that may reflect the user's preferences (by virtue of sharing the at least one characteristic), without necessarily relying on the user of the air purification device 110 to provide any input regarding their preferences.

In some examples, the members of the population group may share a user preference characteristic regarding the content of air in the vicinity of the air purification device. For example, the members of the population group may all have similar preferences regarding smells they like and/or dislike (without necessarily belonging to the same geographical region, age bracket or gender).

The air purification system may be configured to categorise a user of the air purification device into the population group (who share a user preference characteristic regarding the content of air in the vicinity of the air purification device) based on user input in response to the content of air in the vicinity of the air purification device.

The air purification system may be configured to a use a machine learning model to form the population group and to categorise the user into the population group. The machine learning model used to form the population group based on shared user preference characteristics may be a clustering algorithm.

Thus, machine learning can be used to form a population group having shared user preference characteristics with the user of the air purification device, and then this group used to train the machine learning model used by the air purification device. This helps to accelerate the training of the machine learning model in accordance with the preferences of the user of the air purification device, without having to wait for the user of the air purification device to provide statistically meaningful volumes of training data to the network server.

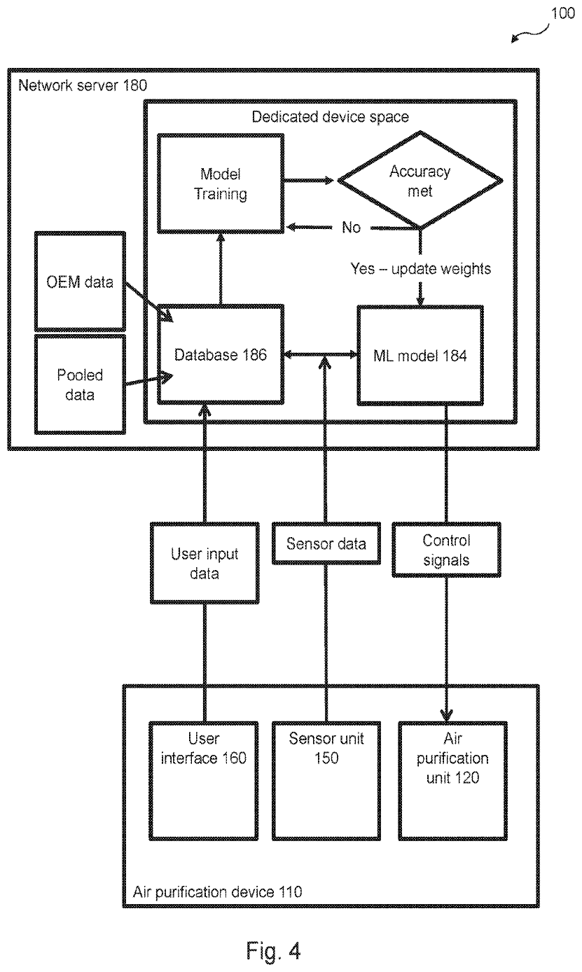

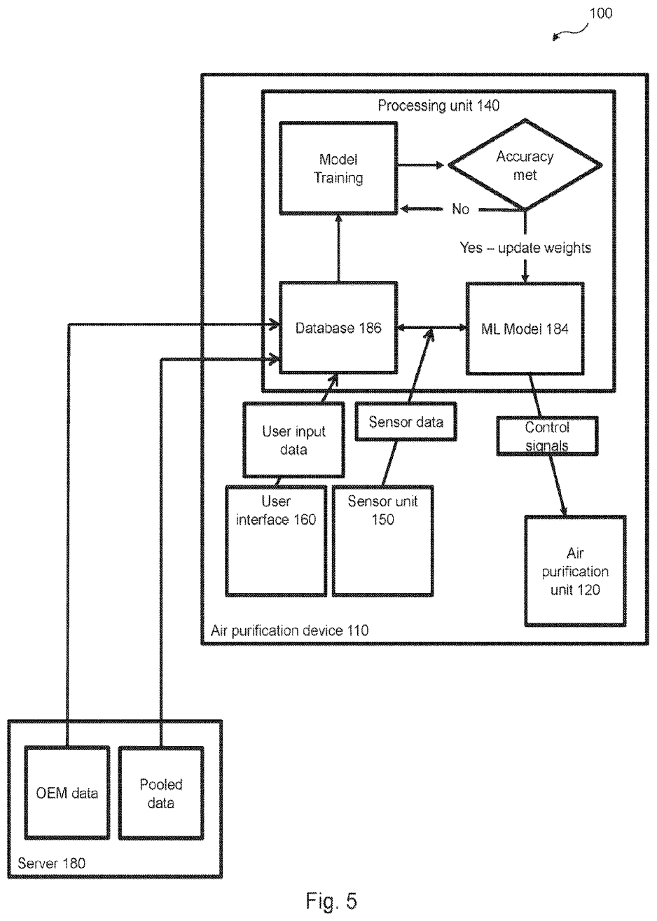

In the example of FIG. 3 (unlike the example of FIG. 4 discussed below), the air purification device can continue to operate even if the wireless communication interface of the air purification device is unable to communicate with the wireless communication interface of the server. Moreover, and unlike the example of FIG. 5 discussed below, the processing required to (re-)train machine learning model is offloaded to the network server.

In the example of FIG. 4, the machine learning model 144 used as part of the control algorithm is located at the network server, and the network server 180 is configured to instruct the air purification unit to operate according to an output of the machine learning model 144.

The steps associated with training the machine learning model here are much the same as with the example of FIG. 3. Except that in the example of FIG. 4, the machine learning model 144 stored at the network server receives sensor data from the sensor unit of the air purification device, makes a determination as to whether (or not) to operate the air purification unit 120, and then sends control signals to the air purification unit to control operation of the air purification unit based on the output of the machine learning model stored at the network server.

In the example shown in FIG. 5, the machine learning model used as part of the control algorithm is located at the air purification device, and the network server is configured to update the control algorithm to change how the air purification device operates the air purification unit by sending training data to the air purification device so that the air purification device updates the machine learning model using the training data. In this example, the training works the same was as in the example of FIG. 3.

The air purification device needs to do much more processing in the arrangement of FIG. 5, compared with the arrangement of FIG. 3, and also requires OEM and pooled data to be sent directly to the air purification device. Whereas the example of FIG. 3 strikes a good balance between resilience against network issues and minimising the processing needed at the air purification device.

For the examples of FIGS. 3 and 4, each air purification device 110, 110A-N may have its own machine learning model 184 stored at the network server, with each purification device 110, 110A-N being trained differently according to one or more characteristics of the user of that purification device 110, 110A-N. This isn't applicable to the example of FIG. 5, in which each air purification device 110, 110A-N would host its own machine learning model.

In some implementations, more than one of the arrangements of FIGS. 3-5 may be used within the air purification system based on the specific air purification device. For example, air purification devices with lower processing power may use the arrangement of FIG. 3 or preferably of FIG. 4, while air purification devices with higher processing power may use the arrangement of FIG. 5. Where the connection quality between the server and the air purification device is poor, the air purification device may be configured to not use the arrangement of FIG. 4. In this way the most appropriate operating mode can be utilised for each of the air purification devices of the air purification system.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

### REFERENCES

A number of publications are cited above in order to more fully describe and disclose the invention and the state of the art to which the invention pertains. Full citations for these references are provided below. The entirety of each of these references is incorporated herein.
WO2019/074256
GB2568979A

1. An air purification system including:
an air purification device; and
a network server;
wherein the air purification device includes:
an air purification unit configured to be operated to purify air in the vicinity of the air purification device; and
a communication interface, wherein the air purification device is configured to communicate with the network server via the communication interface;
wherein the air purification device is configured to operate the air purification unit according to a control algorithm that uses sensor data representative of the content of air in the vicinity of the air purification device as an input;
wherein the network server is configured to update the control algorithm to change how the air purification device operates the air purification unit according to sensor data representative of the content of air in the vicinity of the air purification device.

2. The air purification system according to claim 1, wherein the network server is configured to update the control algorithm based on sensor data associated with user input data indicative of user preferences regarding the content of air in the vicinity of the air purification device.

3. The air purification system according to claim 2, wherein the sensor data used to update the control algorithm includes pooled sensor data associated with multiple users, wherein the multiple users include at least one user other than a user of the air purification device.

4. The air purification system according to claim 3, wherein the multiple users belong to a limited population group who share at least one characteristic with a user of the air purification device.

5. The air purification system according to claim 4, wherein members of the population group share a geographical location characteristic.

6. The air purification system according to claim 4, wherein members of the population group share an age characteristic.

7. The air purification system according to claim 4, wherein members of the population group share a gender characteristic

8. The air purification system according to claim 4, wherein members of the population group share a user preference characteristic regarding the content of air in the vicinity of the air purification device.

9. The air purification system according to claim 8, wherein the air purification system is configured to categorise a user of the air purification device into the population group based on user input in response to the content of air in the vicinity of the air purification device.

10. The air purification system according to claim 8, wherein the air purification system is configured to a use a machine learning model to form the population group and to categorise the user into the population group.

11. The air purification system according to claim 1, wherein:
the control algorithm includes a machine learning model;
the network server is configured to update the control algorithm by updating the machine learning model using training data.

12. The air purification system according to claim 1, wherein:
the air purification unit includes a fan assembly to draw air into the air purification device, and a purification assembly configured to purify air drawn into the air purification unit by the fan

13. The air purification system according to claim 1, wherein:
the air purification device is for use in a building.

14. The air purification system according to claim 1, wherein:
the air purification system includes a sensor unit configured to provide the sensor data representative of the content of air in the vicinity of the air purification device.

15. An air purification device, comprising:
an air purification unit configured to be operated to purify air in the vicinity of the air purification device; and
a communication interface, wherein the air purification device is configured to communicate with a network server via the communication interface;
wherein the air purification device is configured to operate the air purification unit according to a control algorithm that uses sensor data representative of the content of air in the vicinity of the air purification device as an input;
wherein the air purification device receives an updates to the control algorithm from the network server to change how the air purification device operates the air purification unit according to sensor data representative of the content of air in the vicinity of the air purification device.
